(19) United States
(12) Patent Application Publication
(10) Pub. No.: US 2004/0260311 A1
(43) Pub. Date: Dec. 23, 2004

(54) Non-invasive retention device for radiotherapy treatment

(76) Inventors: Philippe Bourel, Genech (FR); Jean Michel Caudrelier, St Vincent De Tyrosse (FR); David Gibon, Lille (FR); Jean Rousseau, Tours (FR)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/489,849

(22) PCT Filed: Sep. 17, 2002
(86) PCT No.: PCT/FR02/03169

(30) Foreign Application Priority Data
Sep. 17, 2001 (FR) 01/12013

(51) Int. Cl. A61B 19/00

(52) U.S. Cl. 606/130

(57) ABSTRACT

The non-invasive retention device for radiotherapy treatment, which can be used in medical imaging, particularly for locating cerebral lesions, comprises:

a first arch (1);

first retention means (2) of said first arch (1) on the patient which are to be positioned at the level of each of the ears of said patient;

second retention means (3) of the first arch on the patient, to be positioned at the level of the nose of the patient;

first locating means (5) which can be used in medical imaging;

means (7, 7c) for fixing the first arch on a table of medical equipment.

The first and second locating means (5; 6) each comprise at least three locating elements (5a; 6a), said locating elements (5a) of the first locating means defining two perpendicular straight lines (D1; D2) and intersecting at a point called centre (O), said locating elements (6a) of said second locating means also defining two perpendicular straight lines (D3; D4) and intersecting with said point known as centre (O), said point known as centre (O) corresponding to the centre of the reference frame attached to the patient.

The present invention relates to a non-invasive retention device for radiotherapy treatment, which can also be used in medical imaging for localising a lesion to be treated or healthy organs to be avoided.

Radiotherapy is used as much for the treatment of cancerous tumours as it is for the treatment of non-tumorous and non-cancerous lesions of type, for example, arterio-veinous malformations. Radiotherapy treatment consists of delivering in one or more sessions a dose of radiation exposure to the lesion to be treated, referred to as the target, by one or more radiation exposure beams. It is thus important that the patient always maintains, from one session to the next, the same set position so that the radiation exposure effectively reaches the target while sparing as far as possible healthy adjacent tissue. To accomplish this, retention systems have been developed to guarantee reproducibility of the positioning of the patient during different radiotherapy sessions, and for each radiation beam.

There are two types of retention system: invasive retention systems and non-invasive retention systems.

Invasive retention systems comprise retention elements, which cooperate with screws, or other types of fasteners implanted directly in the bony table of the cranium of the patient. They are generally utilised in the case of high-dose radiation exposures in a single session within the framework of treatment commonly known as stereotaxic radiosurgery.

Non-invasive retention systems are external systems, which are positioned on the head of the patient without a direct grip on the cranial enclosure. The invention relates to this second type of retention system.

The lesions to be treated are detected by one or more medical imaging techniques, such as for example numeric tomographic imaging (TDM), Magnetic Resonance Imaging (MRI) or Positron Emission Tomography (PET) which enable the cerebral structures of the patient to be reconstructed and visualised in three dimension.

The lesion detected in imaging is first localised spatially relative to a reference frame connected to the imaging device (reference frame imagery). The radiotherapy equipment itself has its own reference frame (reference frame treatment) whereof the centre is located at the isocentre of the equipment, corresponding to the point of concordance of the different beams of radiation exposure. The isocentre of the equipment is, in general, formed by the intersection of luminous beams emitted by wall-mounted positioning lasers (2 lateral and 1 longitudinal).

Radiotherapy treatment is implemented by aligning the centre of the target detected in imaging with the isocentre of the radiotherapy equipment, implying a change of reference frame.

To make this change of reference frame, the most widely used method consists of utilising a reference frame attached to the patient, which allows the position of the target in the imaging reference frame to pass to its position in the treatment reference frame. This method consists of providing the patient reference frame by identifying, on the skin of the patient or on the retention system, points coming from projection of the luminous beams emitted by lasers, which serve to provide the isocentre. On these points, it is possible to position markers visible in imaging whereof the positions can be known in the imaging reference frame. Knowing the position of the target relative to the markers enables the position of the target in the treatment reference frame to be known.

The positioning of this target at the isocentre of the treatment equipment is done by displacing the patient reference frame—and thus the patient him self—to guide the target to the isocentre.

The method providing points on the skin of the patient does not ensure retention of the patient during treatment. Known non-invasive retention systems are often unsuitable for simultaneous use in medical imaging and in radiotherapy.

Accordingly, the device described in FR 2 571 605 effectively allows particularly well-supported non-invasive restraint since the patient is kept at the level of ears and nose on a pedestal equipped with a cushion, and it seems adaptable to numerous morphologies. It allows control of repositioning by using graduations and location of the lesion by imaging due to the visualisation of two triangular structures placed laterally on the head of the patient. However, placing location rods imposes constraints in imaging positioning and the overall bulkiness of the system does not allow optimal use in all imaging devices. In the same way, this device, which is designed so as to allow surgical treatment of the lesion, proves poorly adapted for use in radiotherapy. In effect, the patient reference frame defined by this device is not an orthonorm reference frame centred like that of radiotherapy equipment. As a consequence, it is very difficult to pass from the patient reference frame to that of the radiotherapy equipment without using an information system dedicated especially to this task. It is also very difficult to have these reference frames coincide by the abovementioned laser technique due to the configuration of the device and the materials utilised. In addition, the materials utilised (plastic and metal) absorb radiation, and perturb the dose delivered to the target. Finally, in the device being described, the retention means at the level of ears are fixed on the location bars and controlling the device on the head of the patient is done by deforming a retention arch bearing the retention means at the nose level, and by detrimentally modifying the position in the space of the location bars.

Other devices, such as for example, that described in U.S. Pat. No. 5,531,229 maintain the patient in a set position particularly by means of a buccal positioning element, which the patient keeps gripped between his teeth. If the reproducibility of the positioning is good due to the fabrication of customised buccal positioning elements, restraint can be supported with difficulty and does not allow the patient to communicate. Positioning during radiotherapy treatment implies the addition of plates at the ear level of the patient, while the laser beams providing the reference frame of the treatment equipment strike these plates, allowing positioning of the lesion previously detected in this reference frame. This device is too cumbersome for use in certain imaging equipment such as for example in the head antenna of MRI equipment, this antenna being particularly narrow in the interests of increasing imaging and thus location precision.

In patent application WO 98/55018 a stereotaxy device provided for use in different types of imaging has already been proposed. However, this device can be used only in medical imaging, and is not a retention device for use in radiotherapy; in particular, it does not allow the head of the patient to be immobilised in a predefined and reproducible position on the table of radiotherapy equipment.

The aim of the present invention is thus to propose a non-invasive retention device which can be used both for radiotherapy treatment and for location in medical imaging, and which does not have the disadvantages of the device described in the abovementioned French patent application FR 2 751 605.

This aim is achieved by means of the retention device of claim 1.

According to the present invention, since the first arch, which serves as support to the first and second means of localisation found respectively in imaging and in radiotherapy, is rigid, no deformation of this arch is possible, which would result in a change in position of these locating means relative to the head of the patient, between two treatment sessions or between imaging locating operation and radiotherapy treatment. Furthermore, since the first and second location means defining two reference frames having the same centre corresponding to the centre of the reference frame attached to the patient, it is thus very easy, without software specific to this task, to pass from the reference frame used in imaging (and which thus depends on the first locating means) to that used in radiotherapy (and which thus depends on the second locating means), with the relative position of the first locating means and of the second locating means being perfectly well-known and determined.

The device according to the present invention is not very bulky due to the use of particular retention and locating means, enabling it to be used in all existing imaging equipment and even in the head antenna of MRI equipment.

In addition, since the first locating means are placed at the level of the first arch, they are thus close to the cranium of the patient, allowing the use in imagery of reduced viewing fields in order to limit possible geometric deformations and images of greater definition to be used.

Such necessary retention of the patient on the radiotherapy table or in the imaging equipment can be assured in any known manner. According to a particular embodiment, the retention device according to the present invention comprises a pedestal suitable to be fixed onto the table of medical equipment and in particular of radiotherapy equipment and fixing means of the first arch on the pedestal. Use of such a pedestal ensures restraint of the patient on the radiotherapy treatment table, and thus guarantees fixed and reproducible positioning of the patient throughout treatment.

The positioning and mode of fixing the first and second locating means are not limited according to the present invention. Therefore, the first means and/or the second locating means can be solid with the first arch or placed on a mobile support cooperating with this first arch.

So, according to a variant embodiment, the inventive device comprises a second arch cooperating in a removable way with the first arch, this second arch comprising the location elements of the first locating means and/or of the second locating means. This second mobile arch allows the first locating means used in imaging to be changed easily, which allows the inventive device to be used in different imaging equipment without modifications to its structure.

The present invention, its characteristics and the different advantages it provides will emerge more clearly from the following description with reference to the attached drawings showing a particular embodiment, by way of non-limiting examples and in which:

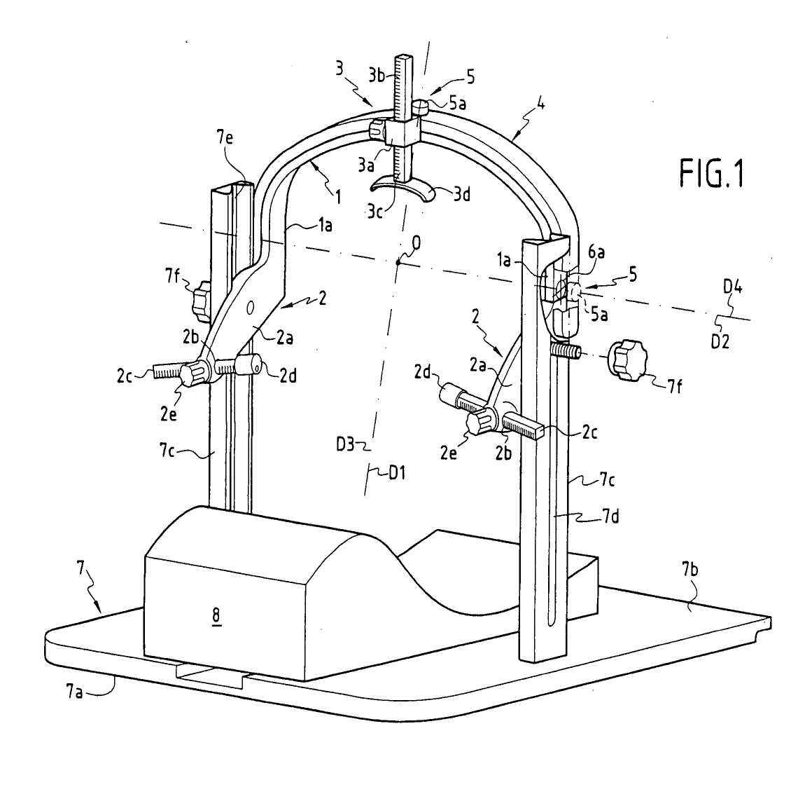

FIG. 1 is a perspective view of a particular embodiment of the device of the invention; and

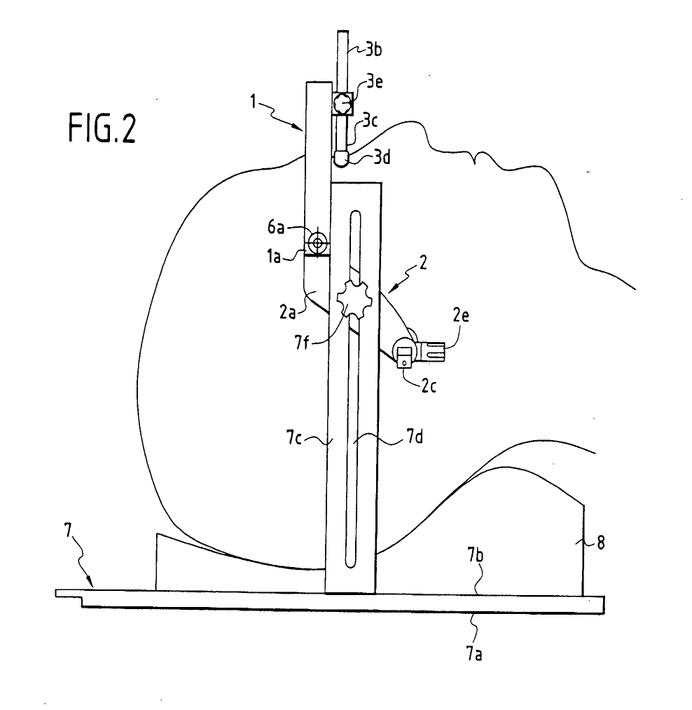

FIG. 2 shows the device of FIG. 1 placed on the head of a patient.

With reference to FIG. 1, the inventive device comprises a first rigid arch 1 whereof the two ends 1a are equipped with first retention means 2 of the arch 1 on the head of the patient. These first retention means 2 comprise a plate 2a fitted with a hole 2b through which passes a hollow rod 2c open at its two ends. The hollow rod 2c cooperates with the hole 2b so as to allow the length of the rod 2c reaching towards inside the first arch 1 to be adjusted. The hollow rods 2c are fitted at one of their ends with a perforated plug 2d. The hollow rod, and more particularly the plug 2d, is intended to abut against the internal wall of the auditory canal of the patient. The rods 2c being hollow and open at their two ends and the plugs 2d being perforated, there is thus no excess pressure on the eardrum of the patient, which might cause him some discomfort. In addition, the auditory capacity of the patient is not excessively altered, when the retention arch 1 is placed on his head.

The rods 2c are graduated so as to allow precise repositioning and are fixed to the plate 2a by means of a screw 2e which either allows the rod 2c to slide into the hole 2b or allows the position of the rod 2c to be gripped in the hole 2b.

In another embodiment, each hollow rod could also comprise a longitudinal internal canal terminating only at one of its two ends (the end to be introduced to the auditory canal), said longitudinal canal also communicating with the exterior via one or more discharging canals, which extend transversally to the longitudinal canal and which act as vents.

Second retention means 3 for cooperating with the bone of the nose of the patient are placed substantially in the axis of symmetry of the first arch 1. These second retention means comprise a support 3a solid with the first arch 1, extending in a plane tangential to the upper part of the first arch and in a direction substantially perpendicular to the latter and a rod 3b which cooperates with the support 3a. The end 3c of the rod 3b is fitted with a nasal support 3d obtained by moulding a customised imprint on the nose of the patient. Since the nasal support 3d is customised as a function of the morphology of the patient, it ensures reliable positioning of the inventive device on the patient. The rod 3b is graduated for precise repositioning and it is fastened to the support 3a by means of a screw 3e, which fixes the rod 3b in position.

The embodiment particular shown in FIG. 1 also comprises a rigid and mobile second arch 4, which cooperates with the first arch 1. This second mobile arch 4 comprises first locating means 5 which can be used in imaging. In the particular case shown here these first locating means 5 comprise three localised locating elements 5a, made of a suitable material, for example, for TDM localisation, of the liquid type rich in protons for MRI or of the contrast product type for PET. These three localising elements 5a are placed according to two perpendicular straight lines D1 and D2 intersecting at point O, which corresponds to the centre of the reference frame attached to the patient (and thus to the retention inventive device). In the particular example shown, an element 5a is placed in the middle of the arch 4, while the two others are placed respectively at each of the ends of this arch.

In the particular embodiment shown, the two arches 1 and 4 cooperate by nesting. The second arch 4 comprises three snugs (not shown), which snap-lock in three openings (not shown) arranged in the first arch 1. The two arches 1 and 4 are superposed perfectly on one another when they are fixed to one another, thus forming a rigid ensemble.

The first arch 1 also comprises second locating means 6 which can be used during the radiotherapy session. In the particular example shown, these second locating means 6 comprise three crosses 6a marked on the surface of the first arch 1. These three crosses 6a are placed according to two straight perpendicular lines D3 and D4 and intersecting at point O as above. In particular the example shown, the straight lines D1 and D2 blend with the straight lines D3 and D4. Accordingly, in the example shown, a cross 6a is placed at the middle of the arch 1 while two other crosses are placed respectively at each of the ends of the latter. The placing of the second locating means 6 is not limited to the invention. Therefore the expert is able to adapt this arrangement as a function of the position of the lasers in the radiotherapy clinic. In particular, it is possible to equip for example not the first arch 1 itself, but for example the rod 3b of a cross 6a or of any other location element utilisable during radiotherapy treatment.

The device according to the present invention also comprises a pedestal 7 whereof the lower surface 7a is to be fixed, for example by means of screws (not shown) or placed on the radiotherapy table. The second face 7b is equipped with a cushion 8 whereof the shape is adapted to immobilise the neck of the patient. Two parallel posts 7c prolong the pedestal 7 in a plane perpendicular to the second face 7b. Each of the posts 7c is in fact a hollow rail whereof the face located opposite the cushion 8 is provided with an elongated hole 7d. A guide 7e solid with the frame 1 cooperates with the hole 7d. The plate 1a comprises a slight protuberance 1c which is embedded in the rail formed by the post 7c and which blocks the arch 1 from rotating while allowing it to be guided by translation along the hole 7d. The position of the first arch is adjusted by sliding the guide 7e in the hole 7d. The blockage of the arch 1 is created by the pulley 7f, which is screwed onto the guide 7e and abuts against the post 7c at the level of the edges of the hole 7d, thus keeping the first arch 1 in position. The presence of the posts 7c on the one hand part ensures effective retention of the patient on the treatment table while avoiding forming a screen to radiation. Cooperation of the first arch 1 with the posts 7c takes into account the anatomy of the patient while allowing height adjustment of the position of the first arch 1 relative to the pedestal 7.

With reference to FIG. 2, when the device of the invention is fixed on the patient, the hollow rods 2c cooperate with the auditory canals of the latter, while the nasal support 3d cooperates with his nose. These two retention means ensure the fixing of the first arch 1 on the head of the patient without deformation of the arch 1.

The use of the device of the present invention will now be described in greater detail.

To locate the lesion in medical imaging, the patient fitted with a first arch 1 on which is fixed the second arch 4 is introduced, for example, in the antenna head of an MRI device. The second mobile arch 4 equipped with a location element adapted to the imaging device used is previously fixed on the first arch 1. According to the type of imaging device utilised, it is also possible to use the two arches 1 and 4 fixed on the pedestal 7. In a device of the scanner type, for example, it is possible to utilise the abovementioned pedestal 7. The lesion is located in three dimension and its coordinates can be calculated in the reference frame attached to the inventive device, that is, the reference frame (O, D1, D2). The graduations of the rods 2c and 3b are noted.

For radiotherapy treatment, the patient is laid out on the radiotherapy table, head on the cushion 8. The first arch 1 is left free to slide along the posts 7c. The second arch 4 can be withdrawn at any time. In the first instance, the first arch 1 is repositioned exactly on the patient by placing the hollow rods 2c and the rod 3b at the same graduations as previously noted. The position of these elements must be exactly the same as those occupied during the location stage in medical imaging. The first arch 1 is then fixed on the posts 7c of the pedestal 7 by means of each of the abovementioned pulleys 7f. The treatment table with the patient is then shifted so that the three laser positioning beams, which equip the radiotherapy clinic, strike each of the three crosses 6a. When this operation is carried out, it is known that the point which corresponds to the intersection of the three laser beams and which thus corresponds to the isocentre of the radiotherapy equipment coincides with the centre O of the reference frame (O, D3, D4). Since the coordinates of the lesion in the reference frame (O, D1, D2) are known, and since the coordinates of the lesion in the reference frame (O, D3, D4) can be calculated simply and without specific calculation software, it is easy to shift the patient so that the point of intersection of the three laser beams coincides with the lesion to be treated in the reference frame (O, D3, D4). The height of the first arch, which is noted on the posts 7c of the pedestal 7, is noted and allows rapid repositioning during different sessions. In the present case, since the straight lines D1 and D2 are merged respectively with the straight lines D3 and D4, the coordinates of the lesion are the same in the two reference frames.

In the event where the abovementioned straight lines are not merged, the change of reference frame is facilitated, as it depends solely on the relative position of the locating elements 5a and 6a which are fixed on rigid arches are not likely to be modified, particularly for example, by mechanical deformation of the device during its use.

I1 is also possible according to the present invention to equip the first arch 1 with locating means locatable in imaging, the second locating means used in radiotherapy able to be placed either on this same first arch or on a second mobile arch.

1. A non-invasive retention device for radiotherapy treatment which can be used in medical imaging, particularly for locating cerebral lesions, said device comprising:
a first arch;
first retention means of said first arch on the patient which are to be positioned at the level of each of the ears of said patient;
second retention means of the first arch on the patient, to be positioned at the level of the nose of the patient;
first locating means which can be used in medical imaging;
means for fixing the first arch on a table of medical equipment;
said device is characterised in that it comprises second locating means which are locatable during radiotherapy, in that the first arch is rigid and serves as support to the first and second locating means, said first and second locating means each comprise at least three locating elements, said locating elements of the first locating means defining two perpendicular straight lines and intersecting at a point called centre, said locating elements of said second locating means also defining two perpendicular straight lines and intersecting with said point known as centre, said point known as centre corresponding to the centre of the reference frame attached to the patient.

2. A retention device as claimed in claim 1, characterised in that the fixing means comprise a pedestal, fixing means of said first arch on said pedestal.

3. The device as claimed in claim 1, characterised in that it comprises a second arch which cooperates in a removable way with said first arch, said second arch comprising said locating elements of said first locating means and/or of said second locating means.

4. The device as claimed in claim 1, characterised in that said locating elements of the second locating means are of the cross type.

5. The device as claimed in claim 1, characterised in that the two straight lines defined by said locating elements of said first locating means are merged with the two straight lines defined by said locating elements of said second locating means.

6. The device as claimed in claim 1, characterised in that said second retention means for cooperating with the nose of the patient comprise a nasal support obtained by moulding a customised imprint on the nose of the patient.

7. The device as claimed in claim 1, characterised in that said first retention means comprise hollow rods to be in part driven into the auditory canal of the patient and open so as to avoid excess pressure on the eardrum.

8. The device as claimed in claim 2, characterised in that said fixing means of said arch on said pedestal comprise two lateral posts which cooperate with said first arch so as to allow height adjustment of said first arch relative to said pedestal.

9. The device as claimed in claim 1, characterised in that the first and second retention means allow the first retention arch to be adapted to a patient, without deformation of said arch.

10. The device as claimed in claim 2, characterised in that:
it comprises a second arch which cooperates in a removable way with said first arch, said second arch comprising said locating elements of said first locating means and/or of said second locating means;
said locating elements of the second locating means are of the cross type;
the two straight lines defined by said locating elements of said first locating means are merged with the two straight lines defined by said locating elements of said second locating means;
said second retention means for cooperating with the nose of the patient comprise a nasal support obtained by moulding a customised imprint on the nose of the patient;
said first retention means comprise hollow rods to be in part driven into the auditory canal of the patient and open so as to avoid excess pressure on the eardrum;
said fixing means of said arch on said pedestal comprise two lateral posts which cooperate with said first arch so as to allow height adjustment of said first arch relative to said pedestal;
the first and second retention means allow the first retention arch to be adapted to a patient, without deformation of said arch.

11. The device as claimed in claim 4, characterised in that the two straight lines defined by said locating elements of said first locating means are merged with the two straight lines defined by said locating elements of said second locating means.

12. The device as claimed in claim 6, characterised in that said first retention means comprise hollow rods to be in part driven into the auditory canal of the patient and open so as to avoid excess pressure on the eardrum.

13. The device as claimed in claim 6, characterised in that the first and second retention means allow the first retention arch to be adapted to a patient, without deformation of said arch.

14. The device as claimed in claim 7, characterised in that the first and second retention means allow the first retention arch to be adapted to a patient, without deformation of said arch.
